(19) United States
(12) Patent Application Publication
(10) Pub. No.: US 2013/0330754 A1
(43) Pub. Date: Dec. 12, 2013

(54) SOL-GEL DERIVED SENSOR FOR RAPID DETERMINATION OF HEAVY METALS

(71) Applicants: John Brennan, Dundas (CA); Zakir Hossain, Hamilton (CA)

(72) Inventors: John Brennan, Dundas (CA); Zakir Hossain, Hamilton (CA)

(73) Assignee: MCMASTER UNIVERSITY, Hamilton, ON (CA)

(21) Appl. No.: 13/912,662

(22) Filed: Jun. 7, 2013

Related U.S. Application Data
(60) Provisional application No. 61/656,607, filed on Jun. 7, 2012.

(51) Int. Cl. C12Q 1/34

(52) U.S. Cl. CPC C12Q 1/34
USPC 435/18

(57) ABSTRACT

The present application is directed to a novel approach for rapid, selective and sensitive detection of heavy metals using a solid-phase bioactive lab-on-paper sensor that is ink-jet printed with sol-gel entrapped reagents to allow colorimetric visualization of the enzymatic activity. The bioactive paper assay is able to detect a range of heavy metals, either alone or as mixtures, in as little as 10 min. The paper-based assay was immune to interferences from non-toxic metal ions such as Na⁺ or K⁺, could be used to detect heavy metals that were spiked into tap water or lake water, and provided quantitative data that was in agreement with values obtained by atomic absorption. The paper-based sensor is of value for rapid, on-site screening of trace levels of heavy metals in resource limited areas and developing countries.

### CROSS-REFERENCE TO RELATED APPLICATIONS

The present application claims the benefit of priority from co-pending U.S. provisional application No. 61/656,607 filed on Jun. 7, 2012, the contents of which are incorporated herein by reference in their entirety.

### FIELD

The present application relates to sensors useful for the rapid determination of heavy metals. In particular, the present application relates to enzyme-based paper sensors for the detection of heavy metals using sol-gel as the immobilization technique.

### BACKGROUND

Heavy metals are widely found in natural and contaminated environments. They cannot be easily detoxified via degradation and thus they may persist in the ecosystem for a long time. These metals are carcinogens and can be involved in several human diseases and/or conditions, including for example, Alzheimer's disease, Parkinson's disease, multiple sclerosis, osteoporosis, development disorder, failure of organs such as the heart, kidney and lungs and failure of the immune system. Thus, monitoring of heavy metals in resources such as food and drinking water is very important.

Traditional methods such as inductively coupled plasma atomic electron spectrometry (ICP/AES), inductively coupled plasma mass spectrometry (ICP/MS), atomic absorption spectroscopy (AAS) or wet chemical methods are used to analyze samples for heavy metals. Though these methods are ultra-sensitive and relatively accurate, they can, for example, consume significant time and money, particularly when large numbers of samples require testing.

Sensor devices such as whole cell^{1,2} DNAzyme/AuNP-based and enzyme-based (e,g., urease, cholinesterase, glucose oxidase, alkaline phosphatase, ascorbic oxidase, horseradish peroxidase, L-lactate dehydrogenase, β-galactosidase)³ biosensors have also been reported for the detection of metal ions. The whole cell biosensor is complicated and requires a long time for preparation. The DNAzyme/AuNP-based assay does not cover a wide range of heavy metals. These techniques are also known to be complex and difficult to use, for example in a colorimetric solid phase assay. Although the enzymatic assays have been shown to detect a wide range of heavy metals with greater speed and/or economy than other known analytical methods, almost all of the known enzymatic assays are in solution phase and fluoremetric and thus cannot be used, for example for on-site monitoring.

To date, no studies have been reported that examine the impact of a broad range of heavy metals on the activity of an enzyme for the purpose of developing a solid phase colorimetric biosensing device. Commercially available test kits such as MetPAD™, MetPLATE™ and Microtox™ for detecting heavy metal toxicity are expensive and require a long time for analysis. Thus, there remains a need for the development of techniques which offer, for example inexpensive, simple, rapid, sensitive and portable detection for on-site monitoring of heavy metals in resources such as drinking water and foods.

Recently, bioactive papers have been developed which can run multiple bioassays and controls simultaneously for detecting, for example toxins, disease markers and illicit drugs and for monitoring health¹¹²⁶ These paper-based sensing devices can be simple, portable, inexpensive and disposable and use low sample volumes. They can be applied, for example in point-of-care testing and/or in field analysis, for example in remote locations with limited facilities. However, there are still no paper-based sensors reported to date which are able to detect heavy metals below the maximum allowable limit.

### SUMMARY

A rapid and sensitive enzyme-based paper sensor for the determination of heavy metals using sol-gel as the immobilization technique has been developed. It has been shown that an enzyme that is inhibited by heavy metals, such as β-galactosidase (B-GAL), can be entrapped between two biocompatible silica layers on paper and that the enzyme retains full activity for a practical period of time under suitable storage conditions. The experimental detection data obtained using the sensor of the present application are consistent with those previously reported using conventional instrumental methods. Moreover, the sensor is stable to a wide range of pHs, indicating that the developed sensor can be used for screening of heavy metal ions from environmental samples.

Accordingly, the present application includes a sensor for detecting heavy metals comprising:

(a) an enzyme entrapped within a sol gel material immobilized in one or more sensing zones on a paper substrate; and

(b) a substrate for the enzyme comprised within a sol gel material immobilized on one or more substrate zones on the paper substrate;

wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals.

The present application also includes a method of detecting heavy metals comprising:

(a) an enzyme entrapped within a sol gel material immobilized in one or more sensing zones on a paper substrate; and

(b) a substrate for the enzyme comprised within a sol gel material immobilized on one or more substrate zones on the paper substrate;

wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals,
the method comprising:
i) contacting the sample with the one or more sensing zones;
ii) placing an eluent at a location proximal to the one or more substrate zones such that the eluent travels by lateral flow across the paper substrate, passing first through the one or more substrate zones and then through the one or more sensing zones; and
iii) monitoring the one or more sensing zones for a detectable change,
   wherein a detectable change in the one or more sensing zones in the presence of the sample compared to a control indicates the presence of heavy metals in the sample.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The present application will now be described in greater detail with reference to the drawings, in which:

FIG. 1 shows a schematic diagram of the detection principle of an enzyme-substrate system and three design strategies for a sensor in embodiments of the present application. (a) The chromogenic substrate, CPRG is hydrolyzed by B-GAL to produce a red-magenta colored product. (b) One dimensional sensing strategy in which β-Gal and CPRG were entrapped within sol-gel derived silica materials in the two dashed regions on a Whatman #1 paper strip (0.5×8 cm). A hydrophobic barrier comprising either methyltrimethoxysilane (MTMS) or wax was introduced at the top of the sensing zone to prevent leaching of color. The sensing zone was incubated for 10 min with a drop of sample and was dipped into H₂O. (c, d) Flower shape multiplexed sensing strategies (made using wax printer) in which β-Gal and CPRG were entrapped within sol gel materials in the circular region and arm, respectively. The sensing zones were incubated with a drop of contaminated sample for 10 min. The bottom of the paper sensor (c) is placed into water while a few drops of water were put at the middle of the sensor (d) for lateral flow chromatography. The color intensity was monitored after 5 min incubation time at room temperature.

FIG. 2 shows dose dependent inhibition effects of heavy metals on β-Gal activity obtained using an exemplary sensor of the present application. (a), (b) The color intensity (CI) at each Hg²⁺ concentration and dose-dependent inhibition responses with the lower levels of Hg²⁺ with one-dimensional sensor (a) and multiplexed patterned paper sensor (b). (c) Semi log plot of the data shown in FIG. 2(a). (d-f) Semi log plots of the data obtained from inhibitory effects of Ag⁺, Cu²⁺ and Cd²⁺. Data are means±s.d. of four independent measurements for each concentration.

FIG. 3 shows an interference study with the non-toxic metal ion, Na⁺ using an exemplary sensor of the present application. NaCl (10 μM) was mixed with different concentrations of Hg²⁺ and tested using an exemplary paper sensor of the present application. All points are means±s.d. of four measurements for each concentration.

FIG. 4 shows single and combined effects of Hg²⁺ (0.1 μM), Ag⁺ (0.1 μM), and Cu²⁺ (3 μM) on the activity of 6-Gal using an exemplary sensor of the present application. C1, C2, and C3 are the combinations of Hg²⁺ (0.1 μM) and Cu²⁺ (3 μM), Hg²⁺ (0.1 μM) and Ag⁺ (0.1 μM), and Ag⁺ (0.1 μM) and Cu²⁺ (3 μM), respectively.

FIG. 5 shows exemplary images of comparing detection of substrate in solution and on paper using an exemplary sensor of the present application.

FIG. 6 shows the determination of (a) Hg(II) and (b) Pb(II) spiked into ddH₂O, tap water, lake water, and falls water, along with control data for the respective unspiked samples using an exemplary sensor of the present application. Data are means±s.d. of three measurements.

FIG. 7 shows an graph demonstrating the effect of a chelator on the detection of heavy metals using an exemplary sensor of the present application. 10 mM EDTA was first mixed with high concentrations of several heavy metals separately [(Hg (2.7 ppm), Ag (2.7 ppm), Cu (8 ppm), Cd (12 ppm), Pb (14 ppm), Ni (24 ppm), Cr (15 ppm)] and tested using the exemplary bioactive paper sensor. Data represents the means±s.d. of four measurements for each concentration.

FIG. 8 shows the detection of individual metals from a mixture of metals using a multiplexed bioactive paper sensor in accordance with an exemplary embodiment of the present application. B-GAL and CPRG were immobilized in two negative control assay zones 1 and 7 (Control, C) and one test zone 2, while Zincon®, sodium diethyldithiocarbamate, diphenylcarbazide and dimethylglyoxime were immobilized in the circular regions of assay zones 3, 4, 5 and 6, respectively. The sample containing Hg(II), Cu(II), Cr(VI) and Ni(II) was added to the circular regions of assay zones 3-6, after which lateral flow was performed using water and the sensor was imaged

### DETAILED DESCRIPTION

### I. Definitions

Unless otherwise indicated, the definitions and embodiments described in this and other sections are intended to be applicable to all embodiments and aspects of the application herein described for which they are suitable as would be understood by a person skilled in the art.

As used in the present application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “suitable” as used herein means that the selection of the particular conditions would depend on the specific method to be performed, but the selection would be well within the skill of a person trained in the art. All method or process steps described herein are to be conducted under conditions sufficient to provide the desired result. Unless otherwise indicated, a person skilled in the art would understand that all method conditions, including, for example, solvent, time, temperature, pressure, reactant ratio and whether or not the method should be performed under an anhydrous or inert atmosphere, can be varied to optimize the desired result and it is within their skill to do so.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

By “biomolecule-compatible” it is meant that a material either stabilizes proteins, and/or other biomolecules against denaturation or does not facilitate denaturation.

The term “biomolecule” as used herein means any of a wide variety of proteins, enzymes, organic and inorganic chemicals, other sensitive biopolymers including DNA and RNA, and complex systems including whole or portions of plants, animals, microorganisms and cells. In an embodiment, the term “biomolecule” refers to an enzyme.

The term “sol gel material” as used herein refers to any material prepared using a sol gel process. The sol-gel process is a wet-chemical technique used for the fabrication of both glassy and ceramic materials. In this process, the sol (or solution) evolves gradually towards the formation of a gel-like network containing both a liquid phase and a solid phase. Precursors are metal alkoxides and metal chlorides, which undergo hydrolysis and polycondensation reactions to form a colloid. The basic structure or morphology of the solid phase can range anywhere from discrete colloidal particles to continuous chain-like polymer networks. In an embodiment, the precursors are silicon or titanium alkoxides or chlorides.

The term “organic polyol” as used herein refers to an organic compound having more than one hydroxy or “OH” group. In an embodiment of the present application, the organic polyol is selected from sugar alcohols, sugar acids, saccharides, oligosaccharides and polysaccharides. Simple saccharides are also known as carbohydrates or sugars. Carbohydrates may be defined as polyhydroxy aldehydes or ketones or substances that hydrolyse to yield such compounds. The polyol may be a monosaccharide, the simplest of the sugars or a carbohydrate. The monosaccharide may be any aldo- or keto-triose, pentose, hexose or heptose, in either the open-chained or cyclic form. Examples of monosaccharides that may be used in the present application include, but are not limited to allose, altrose, glucose, mannose, gulose, idose, galactose, talose, ribose, arabinose, xylose, lyxose, threose, erythrose, glyceraldehydes, sorbose, fructose, dextrose, levulose and sorbitol. The polyol may also be a disaccharide, for example, but not limited to sucrose, maltose, trehalose, cellobiose or lactose. Polyols also include polysaccharides, for example, but not limited to dextran, (500-50,000 MW), amylose and pectin and the like. Other organic polyols that may be used include, but are not limited to glycerol, propylene glycol and trimethylene glycol.

The term “aryloxy” as used herein means phenoxy or naphthyloxy wherein, the phenyl and naphthyl groups may be optionally substituted with 1-5 groups, specifically 1-3 groups, independently selected from the group consisting of halo (fluoro, bromo, chloro or iodo), C₁₋₆alkyl, C₁₋₆alkoxy, OH, NH₂, N(C₁₋₆alkyl)₂, NHC₁₋₆alkyl. C(O)C₁₋₆alkyl. C(O)NH₂, C(O)NHC₁₋₆alkyl, OC(O)C₁₋₆alkyl, OC(O)OC₁₋₆alkyl, NHC(O)NHC₁₋₆alkyl, phenyl and the like.

The term “arylalkyleneoxy” as used herein means aryl-(C₁₋₄-oxy wherein aryl has the same meaning as in “aryloxy”. Specifically, “arylalkyleneoxy” is a benzyl or naphthylmethyl group (i.e. aryl-CH₂—O).

By “normal sol-gel conditions” it is meant the conditions used herein to effect hydrolysis and condensation of the sol gel precursors. This includes, in aqueous solution, at a pH in the range of 4-11.5, specifically in the range 5-10, and temperatures in the range of 0-80° C., and specifically in the range 0-40° C., and optionally with sonication and/or in the presence of catalysts known to those skilled in the art, including acids, amines, dialkyltin esters, titanates, etc.

The term “substrate” as used herein refers to any solid support to which biomolecule compatible silica sol gel materials or other chemical entities can be adhered. In an embodiment of the application, the substrate comprises a substantially planar surface, and is made of any material that supports lateral flow of a solution. When the solution is aqueous based, the substrate is hydrophilic in nature. Conversely, when the solution is non-aqueous, the substrate is hydrophobic in nature. For aqueous solutions, therefore, the substrate may be made from, for example, a paper based material. For non-aqueous solutions, the substrate may be made from materials that are naturally hydrophobic, or that have been treated, for example by derivatization with hydrophobic groups, to make them hydrophobic. In the present application, the substrate is made from paper or a paper-based material. In still other embodiments, the substrate is in the shape of a rectangular test strip, with the first and second ends being opposed to each other. In yet another embodiment, substrate is in shape accommodating a substantially circular arrangement of reagent zones (sensing zones and substrate zones).

The term “paper” or “paper-based material” as used herein refers to a commodity of thin material produced by the amalgamation of fibers, typically plant fibers composed of cellulose, which are subsequently held together by hydrogen bonding. While the fibers used are usually natural in origin, a wide variety of synthetic fibers, such as polypropylene and polyethylene, may be incorporated into paper as a way of imparting desirable physical properties. The most common source of these kinds of fibers is wood pulp from pulpwood trees. Other plant fiber materials, including those of cotton, hemp, linen and rice, may also be used. The paper may be hydrophilic or hydrophobic, may have a surface coating, may incorporate fillers that provide desirable physical properties and may be previously modified prior to coating with the ink jet deposited sol-gel materials, by, for example, precoating with a hydrophilic, hydrophobic or charged polymer layer of organic or inorganic origin.

As used herein, the term “immobilized” of “entrapped” or synonyms thereof, means that movement of the referenced component of the biosensor, is restricted.

The term “sample(s)” as used herein means refers to any material that one wishes to assay using the sensor of the application. The sample may be from any source, for example, any biological (for example human or animal medical samples), environmental (for example water or soil) or natural (for example plants) source, or from any manufactured or synthetic source (for example foods and drinks). It is most convenient for the sample to be a liquid or dissolved in a suitable solvent to make a solution. For quantitative assays, the amount of sample in the solution should be known. The sample is one that comprises or is suspected of comprising one or more heavy metals.

The term “heavy metals” as used herein refers to metal elements with a density higher than about 4.5 g/cm³. The sensor and method of the present application is suitable for the detection of almost all heavy metals, primarily including Zn(II), Cu(II), Cd(II), Pb(II), Ag(I), Cr(VI), Ni(II) and Hg(II), in particular the common heavy metals that pose threats to human, e.g., Cd(II), Pb(II), Hg(II), and the like, due to their toxicity. In an embodiment, the sensor and methods of the present application detect the presence of heavy metal ions.

The term “sensing zone” as used herein refers to an area on the sensor where the presence of a metal is detected. In an embodiment, the one or more sensing zones comprise, in order, beginning adjacent to the substrate: (i) a first sol gel material layer; (ii) the enzyme; and (iii) a second sol gel material layer.

The term “substrate zone” as used herein refers to an area on the sensor where the substrate is localized prior to use of the sensor. In an embodiment, the one or more substrate zones comprise, in order, beginning adjacent to the substrate: (i) a first sol gel material layer; (ii) the substrate; and (iii) a second sol gel material layer. Since the substrate is generally a smaller molecule than the enzyme, it can leach out of the sol gel material upon lateral flow of the eluent across the substrate zone(s) and travel with the eluent through the one or more sensing zones.

The term “control” as used herein means a result obtained under identical conditions that are used for a test result, except for an absence of a parameter of interest or a parameter to be studied. In one embodiment of the present application, a control sample is a sample that is known to not contain heavy metals.

The term “substrate” as used here means a compound that is acted upon by an enzyme and such action, or activity, results in a detectable change to the substrate. In an embodiment, the detectable change is a color change.

The term “lateral flow” as used herein means a movement of an eluent across a substrate, for example, by capillary action.

The term “eluent” as used herein means a liquid that is used to move across the substrate by lateral flow and which flow results in movement of the substrate from the one or more substrate zones to the one or more sensing zones. In an embodiment, the eluent is water.

### II. Sensors of the Application

A rapid and sensitive enzyme-based paper sensor for the determination of heavy metals using sol-gel as the immobilization technique has been developed in the studies of the present application. The present studies demonstrated that B-GAL can be printed between two biocompatible silica layers on paper and that the enzyme retains full activity for at least 2 months when stored, for at 4° C. The sensitivity of the constructed biosensor to metal ions was in the order of Hg, Ag, Cu, and Cd with detection limits of 0.01-20 μM. The experimental data of the present studies is consistent with those previously reported using conventional instrumental methods. The sensors of the present studies are stable over a wide range of pH (5-9). The present studies show that the sensors of the present application are useful for the screening of heavy metal ions in environmental samples.

Accordingly, the present application includes a sensor for detecting heavy metals comprising:

(a) an enzyme entrapped within a sol gel material immobilized in one or more sensing zones on a paper substrate; and

(b) a substrate for the enzyme comprised within a sol gel material immobilized on one or more substrate zones on the paper substrate;

wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals.

In an embodiment of the application, the enzyme is any enzyme whose activity is inhibited by a heavy metal. Such enzymes include those enzymes having a cysteine in their active site or a disulfide linkage in their structure and binding of the heavy metal to the sulfur atom(s) disrupts the enzymatic activity of the enzyme. In an embodiment, the enzyme is β-galactosidase (B-GAL).

In an embodiment of the application, the heavy metal is any such metal whose presence in a sample is interest. In an embodiment, the heavy metal is selected from Zn(II), Cu(II), Cd(II), Pb(II), Ag(I), Cr(VI), Ni(II) and Hg(II). In a further embodiment, the heavy metal is selected from Hg(II) and Ag(I).

In an embodiment, the sensor further comprises a hydrophobic barrier located relative to the one or more sensing zones such that leaching of the enzyme and/or substrate from the one or more sensing zones is inhibited. In an embodiment, the hydrophobic barrier is comprised of wax or other suitable inert material, for example a sol gel material prepared by hydrolysis of an alkyltrialkoxysilane (such as methyltrimethoxysilane). If the hydrophobic barrier is wax, it is an embodiment that it is placed on the paper substrate using a wax printer (such as the Xerox Phaser 8560N). If the hydrophobic barrier is a sol gel material, it is an embodiment that it is placed on the paper substrate using inkjet printing. Methods of printing materials, such as silica-based materials, on paper substrates are described, for example, in U.S. Patent Application publication no. US2012/0135437.

In an embodiment of the application the inhibition of the enzyme's activity is detected using one or more of fluorescence, colorimetry, ultra violet spectroscopy and infrared spectroscopy. In a further embodiment, inhibition of the enzyme's activity is detected using colorimetry. For example, the paper substrate is observed for a visual change in color, wherein a change of color in the presence of the sample, that is different from a change of color in a control, indicates that the sample comprises a heavy metal. The color change is detected or observed simple by eye or using instrumentation, for example, the intensity of the color is monitored or captured using a digital camera or a scanner, with the intensity of the resulting images analyzed using a digital software.

It is an embodiment of the application that the sol gel material is any such material that is biocompatible. In an embodiment, biomolecule-compatible sol gel materials are prepared using biomolecule-compatible techniques, i.e. the preparation involves biomolecule-compatible silica precursors and reaction conditions that are biomolecule-compatible. In a further embodiment, the sol gel material is prepared from a sodium silicate precursor solution. The preparation of sodium silicate solutions for use as a sol-gel precursor is known in the art (see for example, Arduini, F.; Errico, I.; Amine, A.; Micheli, L.; Palleschi, G.; Moscone, D. Anal. chem. 2007, 79, 3409-3415).

In still further embodiments, the sol gel is prepared from organic polyol silane precursors. Examples of the preparation of biomolecule-compatible sol gels from organic polyol silane precursors are described in inventor Brennan's co-pending patent applications entitled “Polyol-Modified Silanes as Precursors for Silica”, U.S. patent application publication no. US2004/0034203 filed on Jun. 2, 2003; and “Methods and Compounds for Controlling the Morphology and Shrinkage of Silica Derived from Polyol-Modified Silanes”, U.S. CIP patent application publication no. US2004/0249082 filed on Apr. 1, 2004.

In embodiments of the present application, the organic polyol is selected from glycerol, sorbitol, maltose and dextran. Some representative examples of the resulting polyol silane precursors suitable for use in the methods of the application include one or more of diglycerylsilane (DGS), monosorbitylsilane (MSS), monomaltosylsilane (MMS), dimaltosylsilane (DMS) or dextran-based silane (DS). In embodiments, the polyol silane precursor is selected from one or more of DGS and MSS.

In a particular embodiment of the application, the sol-gel precursors are combined with an additive which causes spinodal decomposition (phase transition) before gelation, to provide macroporous silica matrixes. Macroporous silica can be used to entrap reagents with large molecular weights, i.e. those molecules that are large enough to not leach from the sol gel. Methods of forming macroporous silica, in particular, from polyol-modified silane precursors are described in inventor Brennan's co-pending patent application entitled “Methods and Compounds for Controlling the Morphology and Shrinkage of Silica Derived from Polyol-Modified Silanes”, U.S. CIP patent application publication no. US2004/0249082 filed on Apr. 1, 2004. In particular, the sol-gel precursor is combined with one or more water soluble polymers which causes spinodal decomposition (phase transition) before gelation. By “water soluble” it is meant that the polymer is capable of being formed into an aqueous solution having a concentration effective to result in phase separation occurring before gelation.

In a further embodiment, sol gels are also obtained by using as sol-gel precursors, compounds of Formula I:

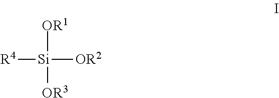

wherein R¹, R² and R³ are the same or different and represent a group that is hydrolyzed under normal sol-gel conditions to provide Si—OH groups; and R⁴ is a group that does not participate directly in the sol gel hydrolysis/polycondensation reaction. Examples of such compounds are described in detail in inventor Brennan's co-pending patent application entitled “Methods and Compounds for Controlling the Morphology and Shrinkage of Silica Derived from Polyol-Modified Silanes”, U.S. CIP patent application publication no. US2004/0249082 filed on Apr. 1, 2004. In embodiments of the application, OR¹, OR² and/or OR³ are the same or different and are derived from organic mono-, di-, or polyols. In embodiments of the present application, the groups OR¹, OR² and/or OR³ are derived from a polyol selected from glycerol, sorbitol, maltose, trehalose, glucose, sucrose, amylose, pectin, lactose, fructose, dextrose and dextran and the like. In further embodiments of the present application, the organic polyol is selected from glycerol, sorbitol, maltose and dextran. In other embodiments of the application, OR¹, OR² and OR³ are the same and are selected from C₁₋₄alkoxy, for example, methoxy or ethoxy, aryloxy and arylalkyleneoxy. In further embodiments of the application, OR¹, OR² and OR³ are all ethoxy. It will be apparent to those skilled in the art that other leaving groups such as chloride or silazane may also be used for the formation of silica according to the methods described in the application.

It should be noted that the groups OR¹, OR² and OR³ are capable of participating directly in the sol gel (hydrolysis/polycondensation) reaction. In particular, these functional groups are alkoxy groups attached to the silicon atom at oxygen, i.e., “Si—OR”, which are hydrolyzed to provide “Si—O—H”, which condense with other “Si—O—H” or “Si—OR” groups to provide “Si—O—Si” linkages and eventually a three-dimensional network within a gel. Trifunctional silanes form silsesquioxanes upon hydrolysis and there is a lower degree of crosslinking in systems derived therefrom, in particular when compared with systems derived from tetrafunctional silanes. The remaining group attached to the silicon atom (R⁴) is a group that generally does not participate directly in the hydrolysis/polycondensation reaction.

Illustrative of compounds of Formula I of the present application, are two classes of the trifunctional silanes based on saccharides which are prepared as described in inventor Brennan's co-pending patent application entitled “Methods and Compounds for Controlling the Morphology and Shrinkage of Silica Derived from Polyol-Modified Silanes”, PCT patent application WO 04/018360, filed Aug. 25, 2003 and corresponding U.S. CIP patent application publication no. US2004/0249082 filed on Apr. 1, 2004.

In further embodiments of the application, the biomolecule-compatible sol gel precursor is selected from one or more of functionalized or non-functionalized alkoxysilanes, polyolsilanes or sugarsilanes; functionalized or non-functionalized bis-silanes of the structure (RO)₃Si—R′—Si(OR)₃, where R may be ethoxy, methoxy or other alkoxy, polyol or sugar groups and R′ is a functional group containing at least one carbon (examples may include hydrocarbons, polyethers, amino acids or any other non-hydrolyzable group that can form a covalent bond to silicon); functionalized or non-functionalized chlorosilanes; and sugar, polymer, polyol or amino acid substituted silicates.

In yet another embodiment of the present application, the biomolecule compatible sol gel precursor solution comprises an effective amount of one or more other additives. In embodiments of the application the other additives are present in an amount to enhance the mechanical, chemical and/or thermal stability of the matrix and/or assay components. In an embodiment, the mechanical, chemical and/or thermal stability is imparted by a combination of precursors and/or additives, and by choice of aging and drying methods. Such techniques are known to those skilled in the art. In further embodiments of the application, the additives are selected from one or more of humectants and other protein stabilizing agents (for e.g. osmolytes). Such additives include, for example, one or more of organic polyols, hydrophilic, hydrophobic, neutral or charged organic polymers, block or random co-polymers, polyelectrolytes, sugars (natural or synthetic), and amino acids (natural and synthetic). In embodiments of the application, the one or more additives are selected from one or more of glycerol, sorbitol, sarcosine and polyethylene glycol (PEG). In further embodiments, the additive is glycerol.

In an embodiment, the precursor solution is prepared according to methods available in the art, for example, for sodium silicate, about 1 g to about 5 g, suitably about 3.0 g, of sodium silicate is dissolved in about 10 mL of doubly distilled water (DDH₂O) followed by addition of about 5 g of Dowex cation exchange resin to replace the sodium ions with protons and stirring until a pH of approximately 4 is reached. The resulting sol is then filtered to remove any fine particulates that could interfere with ink jetting. Suitably, in another embodiment, an organic polyol silane precursor solution is prepared by dissolving about 0.1 g to about 2.0 g, suitably about 1.0 g, of polyol silane, such as DGS, in about 10 mL of ddH₂O, followed by sonication. Again, the resulting sol is then filtered to remove any fine particulates that could interfere with ink jetting. A person skilled in the art would appreciate that if larger scale preparations are required, then the amounts of precursor and water may increase proportionally to provide precursor solutions of approximately the same concentration.

In an embodiment of the application, the sol gel material is deposited on the paper substrate using ink-jet printing of a silica precursor material. As noted above, methods of printing materials, such as silica-based materials, on paper substrates are described, for example, in U.S. Patent Application publication no. US2012/0135437.

After printing of the sensing zone(s) and substrate zone(s) and hydrophobic barrier(s) (if present), the paper substrate is allowed to dry prior to use.

In an embodiment of the application, the one or more sensing zones comprise, in order, beginning adjacent to the substrate: (i) a first sol gel material layer; (ii) the enzyme; and (iii) a second sol gel material layer.

In an embodiment of the application, the substrate zone comprises, in order, beginning adjacent to the substrate: (i) a first sol gel material layer; (ii) the substrate for the enzyme; and (iii) a second sol gel material layer.

In an embodiment of the application, the one or more sensing zones are located above the one or more substrate zones such that the substrate moves from the one or more substrate zones into the one or more sensing zones by lateral flow of an eluent up the paper substrate. In a further embodiment, the one or more sensing zones surround the one or more substrate zones such that the substrate moves from the one or more substrate zones into the one or more sensing zones by lateral flow of an eluent in a concentric fashion out from the one or more substrate zones.

### III. Methods of the Application

The present application also includes a method to detect presence of heavy metals in a sample using a sensor comprising:

a) an enzyme entrapped within a sol gel material immobilized in one or more sensing zones on a paper substrate; and

b) a substrate for the enzyme comprised within a sol gel material immobilized on one or more substrate zones on the paper substrate;

wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals,
the method comprising:

i) contacting the sample with the one or more sensing zones;

ii) placing an eluent at a location proximal to the one or more substrate zones such that the eluent travels by lateral flow across the paper substrate, passing first through the one or more substrate zones and then through the one or more sensing zones; and

iii) monitoring the one or more sensing zones for a detectable change,

wherein a detectable change in the one or more sensing zones in the presence of the sample compared to a control indicates the presence of heavy metals in the sample.

In an embodiment, the sensor further comprises a hydrophobic barrier located relative to the one or more sensing zones such that leaching of the enzyme and/or substrate from the one or more sensing zones is inhibited.

In an embodiment, the method is performed at a pH of about 5 to about 9. In a further embodiment, the method is performed at a pH of about 6 to about 9, or at about 7.5 to about 8.5.

In an embodiment, the sample is contacted with the one or more sensing zones for a period of about 1 minute to about 30 minutes prior to placement of the eluent. In a further embodiment, the sample is contacted with the one or more sensing zones for a period of about 5 minutes to about 15 minutes prior to placement of the eluent.

In an embodiment, the enzyme is B-GAL and the substrate is chlorophenol red β-galactopyranoside (CPRG). In a further embodiment, the concentration of the enzyme is about 100 U/mL to about 300 U/mL or about 200 U/mL. In a further embodiment, the concentration of substrate is about 1 to about 10 mM or about 3 mM.

In an embodiment of the application, the one or more sensing zones are located above the one or more substrate zones such that the substrate moves from the one or more substrate zones into the one or more sensing zones by lateral flow of an eluent up the paper substrate. In a further embodiment, the sensor is in the form of a test strip. In yet another embodiment, the eluent is placed at a location proximal to the one or more substrate zones by placing the bottom end (the end closest to the one or more substrate zones) in the eluent. The eluent, at the beginning of the method does not contact the substrate or sensing zones. In this embodiment, once the eluent comprising substrate travels into the one or more sensing zones, the sensor is removed from the eluent. In an embodiment, the sensor is allowed to dry prior to monitoring any change in the sensing zone, in particular if the change is to be quantitated.

In a further embodiment, the one or more sensing zones surround the one or more substrate zones such that the substrate moves from the one or more substrate zones into the one or more sensing zones by lateral flow of an eluent in a concentric fashion out from the one or more substrate zones.

The following non-limiting examples are illustrative of the present application.

### EXAMPLES

### I. Materials and Methods

### (a) Chemicals and Solutions:

β-galactosidase (B-GAL, grade VIII, from E. coli, EC 3.2.1.23), p-nitrophenyl-β-D-galactopyranoside (PNPG), o-nitrophenyl-β-D-galactopyranoside (OPNG), 5-bromo-4-chloro-3-indolyl-β-D-galactopyranoside (X-Gal), chlorophenol red β-galactopyranoside (CPRG), sodium silicate solution (−14% NaOH, ˜27% SiO₂), dowex 50WX8-100 ion-exchange resin, methyltrimethoxysilane (MTMS), ethylenediaminetetraacetic acid (EDTA), NaCl, MnCl₂.4H₂O, HgCl₂, AgNO₃, CuSO₄ and CdCl₂ were purchased from Sigma-Aldrich. Distilled deionized water (ddH₂O) was obtained from a Milli-Q Synthesis A10 water purification system. All other reagents were of analytical grade.

Stock solutions of enzymes (e.g., B-GAL) and substrates (e.g., X-Gal, PNPG, ONPG, and CPRG) were made up using potassium phosphate buffer (50 mM, pH 7.3). A small amount of Mn²⁺ (final concentration of 0.1 mM) was mixed with enzyme solution for stabilization. These solutions can be used up to three months under appropriate storage conditions (for e.g., storage at −20° C.).

Heavy metal stock solutions were prepared using ddH₂O. EDTA was dissolved in ddH₂O and adjusted to a pH of 7.5 using 1N NaOH. Methyltrimethoxysilane (MTMS) was hydrolyzed by mixing 98% MTMS with 0.1 N HCl in a 5:1 ratio. This mixture was sonicated for 20 minutes on ice to promote ether hydrolysis. All other solutions were prepared using potassium phosphate buffer (50 mM, pH 7.3) if not otherwise stated. All heavy metals are toxic. These materials should be handled, for example with gloves and used in a fumehood.

### (b) Construction of Paper-Based Heavy Metal Sensors:

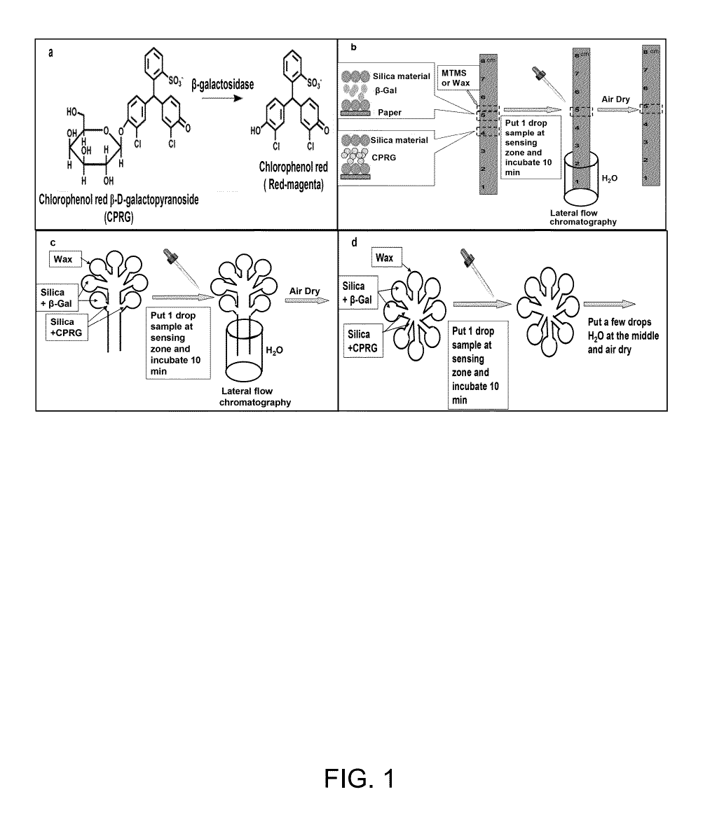

The sensors were constructed based on an enzyme, B-GAL vs. substrate, CPRG system with three design strategies: a one-dimensional strategy and two flower shape strategies. In the one-dimensional strategy, a section of Whatman #1 paper was cut into small pieces (0.5×8 cm) on which B-GAL and CPRG were entrapped using sol-gel derived sol gel materials into two different zones (e.g., enzyme or sensing and substrate zones). To prevent leaching the color, a hydrophobic barrier was also introduced on the top (FIG. 1b) of the sensing zone(s) using either wax (by wax printer, Xerox Phaser 8560N) or methyltrimethoxysilane (MTMS) (via piezoelectric inkjet printing). Both flower shape multiplexed strategies were made using a wax printer in which n-Gal and CPRG were entrapped within sol gel materials in circular regions and arms, respectively as shown in FIGS. 1c, d.

The sensing zones for all cases were incubated with a drop of contaminated sample for 10 min. The bottom of the paper sensor (FIG. 1b, c) was placed into water while a few drops of water were put at the middle of the sensor (FIG. 1d) for lateral flow chromatography. The color intensity was monitored after 5 min air drying at room temperature. The inkjet printing was done as described elsewhere.^{31, 32} After printing, the sensor was allowed to dry for at least 1 h in air at room temperature.

### (c) Optimization of Paper-Based Heavy Metal Biosensor System:

The assay formats were optimized with regard to the enzyme (B-GAL) concentration, type and concentration of substrate, buffer pH, and the time for color development.

The activity of B-GAL concentration on paper was studied. For this, Silica/B-GAL/Silica layers were printed in a width of 0.5 cm across the Whatman #1 filter paper 5 cm from the bottom of the paper (B-GAL concentration range: 0-200 U/mL) as shown in FIG. 1b. The paper strip was then immersed into substrate, CPRG solution (final concentration of 3 mM) and removed as soon as the substrate had reached the enzyme region followed by a certain incubation time (5, 10, and 30 min) at room temperature.

The chromogenic substrates for B-GAL (e.g., PNPG, ONPG, X-Gal, CPRG) assays were tested initially in silica monoliths present in 96 well plates and later on paper. Different concentrations (final concentrations of 0-10 mM) of each of substrate (e.g., X-Gal, CPRG, PNPG, ONPG) were entrapped in silica in a 96 well plate (total volume of 80 μL). 20 μL of B-GAL (final concentration of 100 U/mL) was then added into each well, and the absorbance at 610, 562, 400, and 400 nm was then measured using a TECAN M1000 plate reader after 30 min incubation. A digital camera was also used to take a photograph of the plate.

Different concentrations (final concentration of 0-10 mM) of each of the substrates (e.g., X-Gal, CPRG, PNPG, ONPG) and B-GAL (final concentration of 100 U/mL) were entrapped on paper as described in the previous section (FIG. 1b). The sensor was then immersed into H₂O and removed as soon as the substrate had reached the sensing region followed by a certain incubation time (5, 10, and 30 min) at room temperature.

The effect of pH on enzyme assay was initially investigated in solution. For this, CPRG was dissolved with potassium phosphate buffer (75 mM) having different pH values (6-8.5). 80 μL of CPRG (final concentration of 3 mM) solution and 20 μL of B-GAL (final concentration of 100 U/mL) were mixed into a 96-well plate. A kinetic study for B-GAL (at 562 nm) catalyzed reactions were then performed using a TECAN Safire microwell plate reader for up to 60 min.

The effect of pH on enzyme assay with different incubation time on paper was also studied. For this, Silica/CPRG/Silica layers were printed or over spotted at a width of 0.5 cm across the Whatman #1 filter paper, 5 cm from the bottom of the paper (CPRG concentration of 3 mM), while silica/B-GAL/silica layers were printed or over spotted in a 0.5 cm wide area across the paper strip, 6 cm from the bottom of the paper (B-GAL concentration 100 U/mL) (see FIG. 1b). The sensors were then immersed into buffers having different pH values (6-8.5) and were removed as soon as the buffer had reached to the sensing region via lateral flow. B-GAL hydrolyzes the chromogenic substrate CPRG (yellow in color) to produce chlorophenol red (CPR) (red-magenta in color). The color intensity was then monitored over different incubation times (5, 10, and 30 min).

### (d) Measurement of Heavy Metal Inhibition:

Four heavy metals including Hg²⁺, Ag⁺, Cu²⁺, Cd²⁺ ions were selected in this study. The inhibitory effects of these metals on the solid-phase biosensor were evaluated by measuring the decrease in the color intensity produced by the enzyme-substrate reaction. The sensing region of the paper sensor was first incubated with various concentrations of heavy metals (5 μL) for 10 min after which the paper sensor was allowed for lateral flow chronographic system. The images were taken after 5-10 min air drying at room temperature.

(e) Interference with Nontoxic Metal Ions.

In the interference study, a fixed amount of a solution comprising a nontoxic metal, NaCl (10 μM) was mixed with different concentrations of Hg samples. The sensor was incubated with a 5 μL sample for 10 min (a separate paper sensor was made for each concentration) and the B-GAL inhibition tested as outlined above.

### (f) Data Processing and Statistics.

The color intensity of the sensing areas was monitored either by the naked eye, by obtaining a digital image (Canon G11 12 MegaPixel camera operated in automatic mode with no flash and with the macroimaging setting on) or by using an office scanner, with ImageJ™ software being used to analyze the Jpeg images in the latter two cases. The numerical single color coordinates values were obtained using the ImageJ™ color split channel system. All data points represent the average of four replicate experiments and the error bars represent the standard deviations (s.d.) of those points.

### (g) Validation Study.

Heavy metals were spiked into water samples (15 mL each) at known high and low concentrations and assayed first using the bioactive paper sensor strips, and then by atomic absorption spectroscopy (AAS) using an AAnalyst 400 Atomic Absorption Spectrophotometer with hollow cathodic lamps and a conventional 10 cm slit burner head. Prior to analyzing the sample via AAS, standard curves were obtained for each heavy metal ion using standard solutions containing 3% HCl. Absorbance values were taken after averaging over a one second integration time. The standard curve was constructed by plotting the absorbance values against metal concentration.

### II. Results and Discussion

### (a) Analytical Performance of Heavy Metal Test Strips:

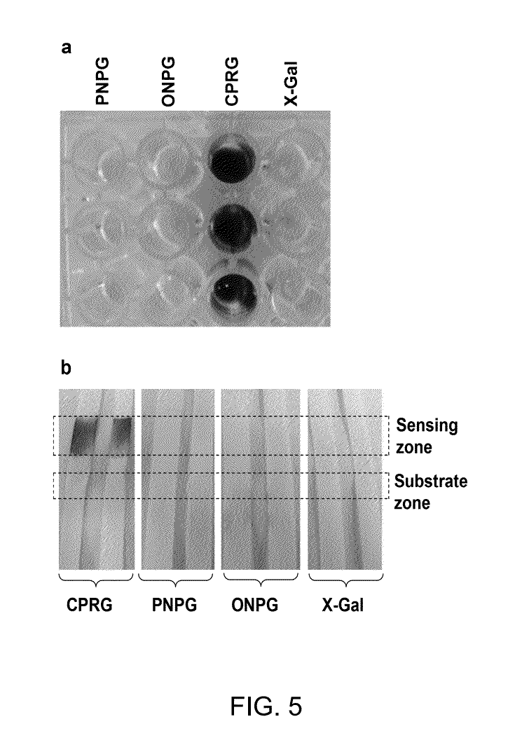

Initial studies involved the optimization of the bioactive paper sensor in terms of types and concentration of substrate, B-GAL concentration, assay pH, and time for color development in order to obtain the best detection limits for a variety of heavy metal ions. These studies revealed that CPRG was the most suitable substrate for the B-GAL assay (see FIG. 5), in agreement with the results of previous comparative studies of different chromogenic B-GAL substrates.³³ The optimum CPRG and B-GAL concentrations for the inhibition reaction were found to be 3 mM and 200 U/mL, respectively, with an assay pH of 8.0 being optimal for enzyme activity. Assays were run at pH 7.3 to minimize the potential for hydroxide formation. Note that pH values less than 5 or higher than 9 resulted in decreases in enzyme activity, and thus will cause a deviation in the results. The color intensity, as determined by digital photography of test strips followed by image analysis with ImageJ, increases with time and is detectable in as little as 1 min. However to make the data consistent, the images were taken after 10 min for all the subsequent experiments and imaged using a flatbed scanner to avoid issues with variations in ambient lighting.

Using optimum conditions, inhibition studies were performed using the bioactive paper sensors developed in the present studies.

It is well-known that heavy metals including Hg, Ag, Cu, and Cd are potent inhibitors of B-GAL due to the reaction of a sulfhydryl group of cysteine in B-GAL with metal ions. To assess whether the paper-based biosensors of the present studies were able to detect pM levels of these metals, assays were conducted by incubating the sensing zone of the test strips with solutions containing various levels of metals for 10 min and then H₂O was used to move substrate, CPRG to the sensing region.

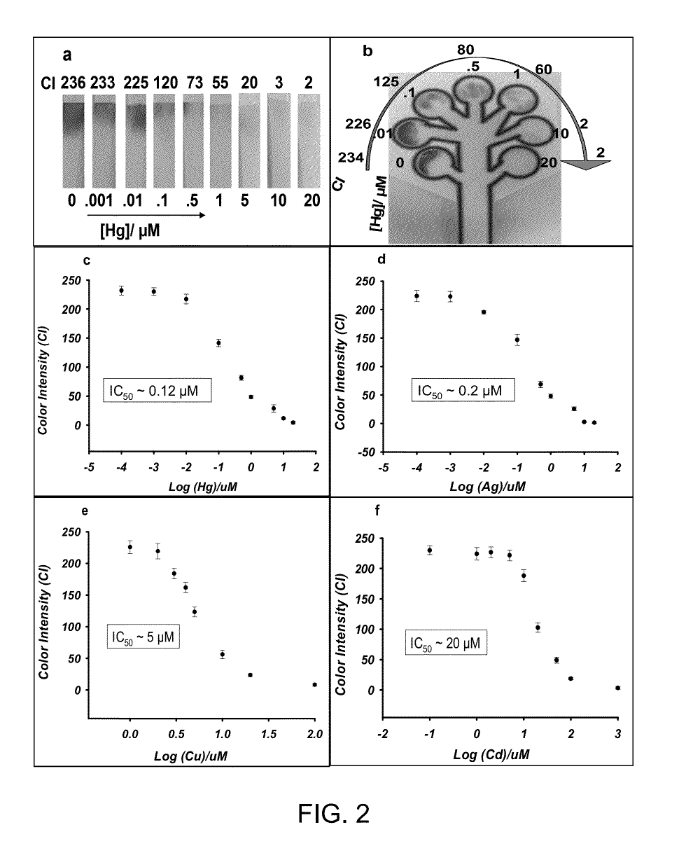

FIGS. 2a-b show the visual color intensity and concentration-dependent inhibition responses of Hg with one dimensional (a) and multiplexed paper sensors (b), and clearly shows a decrease in red-magenta color intensity with increasing metal concentration. FIGS. 2c-f demonstrate the semi log plots of the data obtained from inhibitory effects of Hg²⁺, Ag⁺, Cu²⁺ and Cd²⁺. The data suggest that increasing concentrations of all of these metals progressively inhibit the activity of B-GAL.

On the basis of these results, the limits of detection (LODs) of Hg(II), Ag(I), Cu(II), Cd(II), Pb(II), Cr(VI) and Ni(II) were found to be approximately 0.001, 0.002, 0.020, 0.020, 0.140, 0.150 and 0.230 ppm, respectively, while the IC₅₀ values (concentration to obtain 50% inhibition of B-GAL) were 0.03, 0.05, 0.70, 1.14, 1.40, 1.49, and 1.80 ppm, respectively. respectively. The LODs for all metals are similar to or lower than the maximum allowable limit (MAL) according to WHO guidelines,³⁴ and are well below the MAL provided in the SABS 241 guidelines.³⁵ The results demonstrate that Hg(II) and Ag(I) are the most potent inhibitors of B-GAL, followed by Cu(II)>Cd(II)>Pb(II)=Cr(VI)>Ni(II), which is consistent with the affinity of these metal ions toward the sulfhydryl group.³⁶ These test strips could be stored for at least two months at 4° C. and at least 2 weeks at room temperature without a noticeable decrease in the B-GAL activity.

The present observations are in agreement with the results of previous studies using a spectrophotometer indicating that the present biosensor shows a comparable sensitivity to the expensive spectrophotometer but with benefits such as easy preparation and/or miniaturization.

The data clearly show that the detection of the heavy metals is possible with the naked eye. This permits the use of the test strips directly in the field without sophisticated and/or expensive instrumentation. The presence of a simple colorimetric readout also enables rapid imaging and transmission to a central lab for further quantitative analysis using, for example a standard cell phone camera combined with, for example a means of transmission such as e-mail or MMS messaging.^{13,15,17}

### (b) Interferences on Assay Performance:

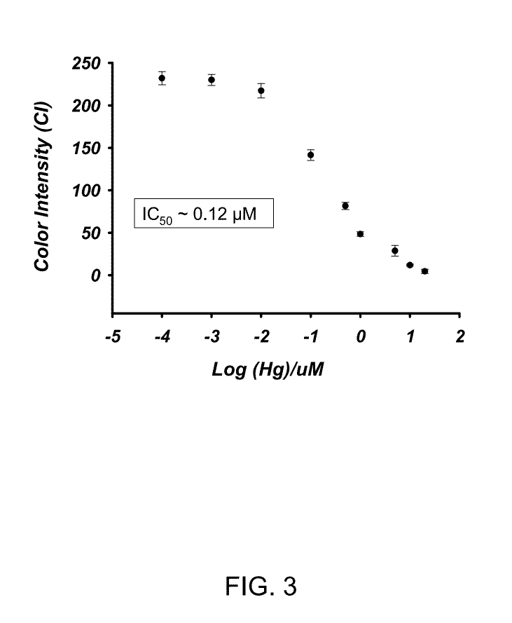

In order to investigate the interferences on the signal by other non-toxic metal ions, a solution comprising a high concentration of NaCl (10 μM) was mixed with different concentrations of Hg²⁺ and tested using paper sensors as detailed above. FIG. 3 shows that there was a negligible effect on the general shape of the response vs. concentration or the limit of detection (˜0.01 μM), with the IC50 value for Hg being 0.12 μM. All the data were highly reproducible. Similar results were obtained with KCl (100 ppm). The assay is therefore useful for monitoring heavy metals in environmental samples.

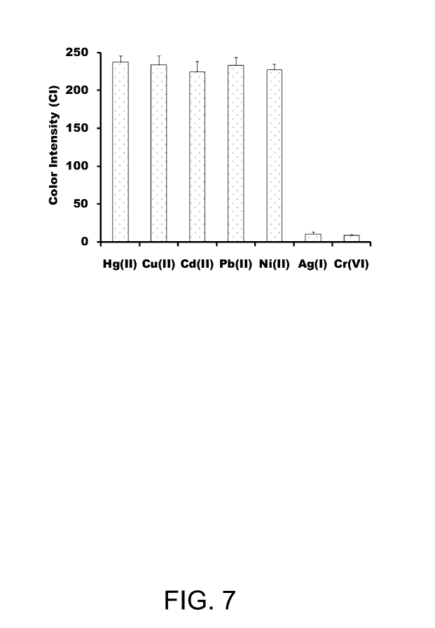

To enhance the ability to quantify heavy metals selectively, EDTA (10 mM) was added to samples containing individual metal ions (Hg(II) and Ag(I) ˜2.7 ppm each; Cu(II), Cd(II), Pb(II), Ni(II), and Cr(VI)˜8 ppm each), followed by testing of the metal-EDTA mixture using the bioactive paper strip. As shown in FIG. 7, the inclusion of EDTA completely removed the inhibition from Hg(II), Cu(II), Cd(II), Pb(II) and Ni(II) (EDTA-metal conditional formation constants at pH 7, K′_{f}>10¹³ M), while retaining the inhibition originating from Ag(I) and Cr(VI) (K′_{f}<10⁴ M). Thus, selective detection of either Ag(I) or Cr(VI) is possible even in the presence of several other metal ions if an appropriate amount of EDTA is utilized to chelate the other ions.

### (c) Testing of Environmental Samples.

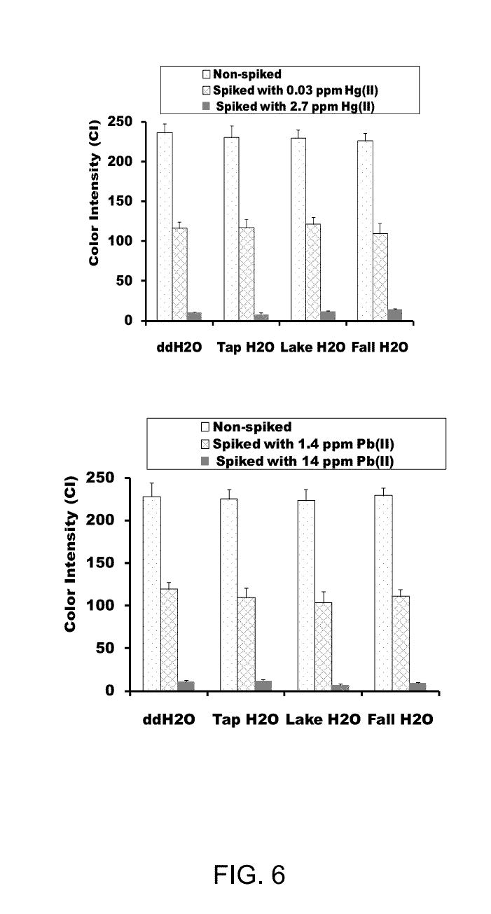

To demonstrate the applicability of the bioactive paper sensor strips for the determination of heavy metals in real environmental samples, distilled water (pH 5.5), tap water (pH 6), lake water (pH 7.5, Bayfront Park, Hamilton, ON, Canada), and water from a local waterfall (pH 7.4, Webster's Falls, Hamilton, ON, Canada) were collected and spiked with high and low levels of each metal ion. ICP-MS analysis of the lake and waterfall samples showed that no metals of interest were present at levels above 0.003 ppm, and all were present at levels below their respective detection limits for the paper based sensor. As shown in FIG. 6, unspiked samples did not provide any inhibition of the B-GAL reaction (CI˜230), demonstrating that all of the sample matrixes were compatible with the assay and that none of these samples contained heavy metals. Samples that were spiked with Hg(II) (FIG. 6a) or Pb(II) (FIG. 6b) resulted in a similar decreases in color intensity regardless of the sample matrix. Hence, it is possible to detect heavy metal ions in all of the sample matrixes, even with pH values varying from 5.5 to 7.5. These results demonstrate that the bioactive paper sensor can be used for rapid analysis of either tap or lake water with no interferences.

### (d) Single and Combined Effects of Heavy Metals on β-Galactosidase Activity:

The single and combined effects of several heavy metals including Hg, Ag, and Cu on the activity of β-galactosidase were investigated with the paper-based metal sensor of the present studies in order to illustrate the mixed effect as well as to further characterize their action mechanisms.

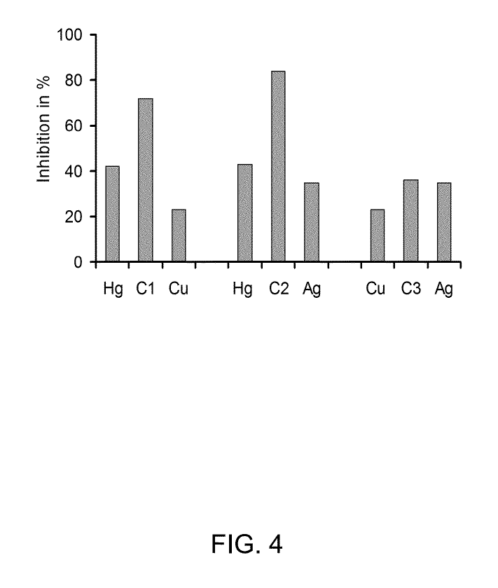

The relative inhibition of BGAL by several combinations of heavy metals was compared with that of the inhibition by each single metal and is shown in FIG. 4. It was found that certain metals act in a synergistic way: 0.1 μM Hg and 3 μM Cu combined (combination C1) cause a 72% inhibition, while the respective value for Cu alone is 23% inhibition, and for Hg alone is 42% inhibition. In contrast, the combination of Hg and Ag (combination C2) results in a simple addition of effects: 0.1 μM Hg causes about 42% inhibition, 0.1 μM Ag about 35% inhibition, and both together about 84% inhibition. The combination of Cu (3 μM) and Ag (0.1 μM) resulted in a similar inhibition (36%) compared to that of Ag inhibition (35%). Cu showed no influence on Ag inhibition. In agreement with this, Tsai et al.³⁷ reported that the combination effect of certain metals, including Cu, Cd, and Zn ions of urease were different. This depicts that the combined effect of heavy metals on the inhibition of enzyme is quite complicated.

### (e) Storage Stability of the Sensor:

The long-term stability of the sensor strips of the present studies was also investigated. These test strips could be stored for at least two months at 4° C. and at least 2 weeks at room temperature without a noticeable decrease in the B-GAL activity.

### (f) Multiplexed Screening of Heavy Metal Ions.

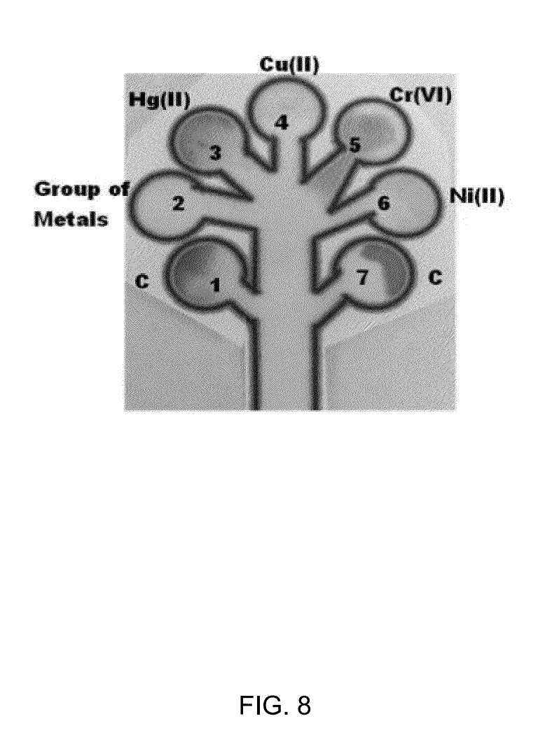

The paper-based metal ion sensor can detect inhibition owing to either individual metal ions or mixtures or metal ions, but the enzyme-based assay does not have the capability to identify which metals are present in a mixture. To address this issue, a multiplexed sensor was prepared that used two assay arms as controls, one for testing a mixture of metal ions using the B-GAL assay, and four additional assay arms that had colorimetric reagents (Zincon®, sodium diethyldithiocarbamate, 1,5-diphenylcarbazide or dimethylglyoxime) that could selectively form complexes with either Hg(II), Cu(II), Cr(VI) or Ni(II), respectively. A sample (5 μL) containing Hg(II), Cu(II), Cr(VI) and Ni(II) at 0.5 ppm each was then added to the sensing regions in assay zones 2-6 and incubated for 10 min. The “stem” of the sensor was then immersed into ddH₂O until the CPRG reached the circular regions of assay zones 1, 2 and 7. The color intensity from each assay zone was monitored after 10 min drying time at room temperature and the results are shown in FIG. 8. In addition to the expected inhibition of B-GAL, each of the colorimetric reagents underwent the expected color change (Zincon®: formation of blue color; sodium diethyldithiocarbamate: formation of brown color; 1,5-diphenylcarbazide: formation of red magenta color, dimethylglyoxime: formation of pink color) in the presence of Hg(II), Cu(II), Cr(VI) and Ni(II), respectively. Absence of any of these metals resulted in no color change from the respective sensing zone, as expected. Although cross-sensitivity issues were not observed in the present assays, it is possible that the indicators may respond to higher levels of the heavy metals that were tested, or to other heavy metals not present in our assays. A point to note is that the colorimetric reagents have substantially higher detection limits (˜0.5-1 ppm) as compared to the B-GAL assay (0.001, 0.020, 0.150 and 0.230 ppm for Hg(II), Cu(II), Cr(VI) and Ni(II), respectively), and thus cannot detect metals at concentrations below the maximum allowable limit. Thus, the B-GAL assay is still required to obtain acceptable detection limits, while the colorimetric reagents can provide identification of metals in highly contaminated samples. It is expected that such sensors could be particularly useful in large scale routine screening of drinking water samples or industrial process streams in developing countries, where the heavy metal concentrations are usually relatively high

### III. Summary

An enzyme-based colorimetric paper sensor fabricated by sol-gel technique and ink-jet printing technology for the rapid and sensitive determination of heavy metals has been developed. In the present examples, the chromogenic substrate, CPRG was hydrolyzed by B-GAL to form a red-magenta colored product. FIG. 1a illustrates the detection principle of a CPRG-based colorimetric assay. The assay protocol for a one-dimensional and two flower-shaped strategies is illustrated schematically in FIGS. 1b-d. The paper-based biosensors allowed for visual, fast, convenient, and concentration-dependent monitoring of heavy metals sensitively. The interferences with nontoxic metals, the combined effects, long-term stability and overall assay performance was also examined. To explore the possibility of a selective means of metal detection in aqueous samples, the ability of EDTA and NTA to form metal complexes and thus prevent B-GAL inhibition by heavy metals was also investigated.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE SPECIFICATION

¹ Lehmann, M.; Riedel, M.; Adler, K.; Kunze, G. Biosen. Bioelectron. 2000, 15, 211-219.
² Tauriainen, S.; Karp, M.; Chang, W.; Virta, M. Biosen. Bioelectron. 1998, 13, 931-938.
³ Tsai, H.-C.; Doong, R.-A.; Chiang, H.-C.; Chen, K.-T. Anal. Chem. Act. 2003, 481, 75-84.
⁴ Senillou, A.; Jaffrezic-Renault, N.; Martelet C.; Cosnier, S. Talanta 1999, 50, 219-226.
⁵ Krawczyk, T. K.; Moszczyñska, M.; Trojanowicz, M. Biosen. Bioelectron. 2000, 15, 681-691.
⁶ Starodub, N. F.; Kanjuk, N. I.; Kukla, A. L.; Shirshov, Y. M. Anal. Chim. Acta 1999, 385, 461-466.
⁷ Kukla, A. L.; Kanjuk, N. I.; Starodub, N. F.; Shirshov, Y. M. Sens. Actuators B 1999, 57, 213-218.
⁸ Satoh, I.; Iijima, Y. Sens. Actuators B 1995, 24(25), 103-106.
⁹ Han, S.; Zhu, M.; Yuan, Z.; Li, X. Biosen. Bioelectron. 2001,16, 9-16.
¹⁹ Fennouh, S.; Casimiri, V.; Geloso-Meyer, A.; Burstein., C. Biosen. Bioelectron. 1998, 13, 903-909.
¹¹ Li, X.; Tian, J.; Nguyen, T.; Shen, W. Anal. Chem. 2008, 80, 9131-9134.
¹² Abe, K.; Suzuki, K.; Citterio, D. Anal. Chem. 2008, 80, 6928-6934.
¹³ Martinez, A. W.; Phillips, S. T.; Butte, M. J.; Whitesides, G. M. Angew. Chem. 2007, 46, 1318-1320.
¹⁴ Zhao, W.; Ali, M. M.; Aguirre, S. D.; Brook, M. A.; Li, Y. Anal. Chem. 2008, 80, 8431-8437.
¹⁵ Bruzewicz, D. A.; Reches, M.; Whitesides, G. M. Anal. Chem. 2008, 80, 3387-3392.
¹⁶ Su, S.; Ali, M. M.; Filipe, C. D. M.; Li, Y.; Pelton, R. Biomacromolecules 2008, 9, 935-941.
¹⁷ Martinez, A. W.; Phillips, S. T.; Whitesides, G. M. Proc. Natl. Acad. Sci. USA 2008, 105, 19606-19611.
¹⁸ Liu, J. et al. Anal. Chem. 2010, 82, 2463-2471.
¹⁹ Apilux, A. et al. Anal. Chem. 2010, 82, 1727-1732.
²⁰ Noh, H.; Phillips, S. T. Anal. Chem. 2010, 82, 4181-4187.
²¹ Khan, M. S.; Thouas, G.; Shen, W.; Whyte, G.; Garnier, G. Anal. Chem. 2010, 82, 4158-4164.
²² Martinez, A. W.; Phillips, C. E.; Thomas III, S. W.; Sindi, H.; Whitesides, G. M. Anal. Chem. 2008, 80, 3699-3707.
²³ Pelton, R. Trends in Anal. Chem. 2009, 28, 925-942.
²⁴ Martinez, A. W.; Phillips, S. T.; Whitesides, G. M. Anal. Chem. 2010, 82, 3-10.
²⁵ Xu Li, X.; Tian, J.; Shen, W. Appl. Mater. Interfaces 2010, 2, 1-6.
²⁶ Lu, Y.; Shi, W.; Qin, J.; Lin, B. Anal. Chem. 2010, 82, 329-335.
²⁷ Zhao, W.; Ali, M. M.; Aguirre, S. D.; Brook, M. A.; Li, Y. Anal. Chem. 2008, 80, 8431-8437.
²⁸ Martinez, A. W.; Phillips, S. T.; Whitesides, G. M. Anal. Chem. 2010, 82, 3-10.
²⁹ Apilux, A. et al. Anal. Chem. 2010, 82, 1727-1732.
³⁰ Luckham, R. E.; Brennan, J. D. Analyst 2010, 135, 2028-2035.
³¹ Hossain, S. M. Z. et al. Anal. Chem. 2009, 81, 5474-5483.
³² Hossain, S. M. Z.; Luckham, R. E.; McFadden, M. J.; Brennan, J. D. Anal. Chem. 2009, 81, 9055-9064.
(³³) Wutor, V. C.; Togo, C. A.; Pletschke, B. I. Chemosphere 2007, 68, 622-627.
(³⁴) http://www.lenntech.com/who-eu-water-standards.htm
(³⁵) http://viront.com/Info_Pages/SANS_{—}241.pdf
(³⁶) Preininger, C.; Wolfbeis, O. S. Biosen. Bioelectron. 1996, 10-11, 981-990.
³⁷ Tsai, H.-C.; Doong, R.-A.; Chiang, H.-C.; Chen, K.-T. Anal. Chem. Act. 2003, 481, 75-84.

1. A sensor for detecting heavy metals comprising:
(a) an enzyme entrapped within a sol gel material immobilized in one or more sensing zones on a paper substrate; and
(b) a substrate for the enzyme comprised within a sol gel material immobilized on one or more substrate zones on the paper substrate;
wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals.

2. The sensor of claim 1, wherein the enzyme is β-galactosidase (B-GAL).

3. The sensor of claim 1, wherein the heavy metal is selected from Zn(II), Cu(II), Cd(II), Pb(II), Ag(I), Cr(VI), Ni(II) and Hg(II).

4. The sensor of claim 1, further comprising a hydrophobic barrier located relative to the one or more sensing zones such that leaching of the enzyme and/or substrate from the one or more sensing zones is inhibited.

5. The sensor of claim 1, wherein inhibition of the enzyme's activity is detected using one or more of fluorescence, colorimetry, ultra violet spectroscopy and infrared spectroscopy.

6. The sensor of claim 5, wherein inhibition of the enzyme's activity is detected using colorimetry.

7. The sensor of claim 1, wherein the sol gel material is a silica material.

8. The sensor of claim 1, wherein the sol gel material is deposited on the paper substrate using ink-jet printing of a silica precursor material.

9. The sensor of claim 1, wherein the one or more sensing zones are located above the one or more substrate zones such that the substrate moves from the one or more substrate zones into the one or more sensing zones by lateral flow of an eluent up the paper substrate.

10. The sensor of claim 1, wherein the one or more sensing zones surround the one or more substrate zones such that the substrate moves from the one or more substrate zones into the one or more sensing zones by lateral flow of an eluent in a concentric fashion out from the one or more substrate zones.

11. A method to detect presence of heavy metals in a sample using a sensor comprising:
a) an enzyme entrapped within a sol gel material immobilized in one or more sensing zones on a paper substrate; and
b) a substrate for the enzyme comprised within a sol gel material immobilized on one or more substrate zones on the paper substrate;
wherein the one or more sensing zones are located relative to the one or more substrate zones such that the substrate moves from the one or more substrate zones into the one or more sensing zones by lateral flow of an eluent across the paper substrate in a direction from the one or more substrate zones to the one or more sensing zones, and
the enzyme's activity is inhibited by heavy metals,
the method comprising:
i) contacting the sample with the one or more sensing zones;
ii) placing an eluent at a location proximal to the one or more substrate zones such that the eluent travels by lateral flow across the paper substrate, passing first through the one or more substrate zones and then through the one or more sensing zones; and
iii) monitoring the one or more sensing zones for a detectable change,
wherein a detectable change in the one or more sensing zones in the presence of the sample compared to a control indicates the presence of heavy metals in the sample.

12. The method of claim 11, wherein the enzyme is β-galactosidase (B-GAL).

13. The method of claim 11, wherein the heavy metal is selected from Zn(II), Cu(II), Cd(II), Pb(II), Ag(I), Cr(VI), Ni(II) and Hg(II).

14. The method of claim 11, wherein the sensor further comprises a hydrophobic barrier located relative to the one or more sensing zones such that leaching of the enzyme and/or substrate from the one or more sensing zones is inhibited.

15. The method of claim 11, wherein inhibition of the enzyme's activity is detected using one or more of fluorescence, colorimetry, ultra violet spectroscopy and infrared spectroscopy.

16. The method of claim 15, wherein inhibition of the enzyme's activity is detected using colorimetry.

17. The method of claim 15, wherein the sol gel material is a silica material.

18. The method of claim 15, wherein the sol gel material is deposited on the paper substrate using ink-jet printing of a silica precursor material.

19. The method of claim 11, wherein the one or more sensing zones are located above the one or more substrate zones such that the substrate moves from the one or more substrate zones into the one or more sensing zones by lateral flow of the eluent up the paper substrate.

20. The method of claim 11, wherein the one or more sensing zones surround the one or more substrate zones such that the substrate moves from the one or more substrate zones into the one or more sensing zones by lateral flow of the eluent in a concentric fashion out from the one or more substrate zones.
